(19) United States
(12) Patent Application Publication
(10) Pub. No.: US 2008/0228262 A1
(43) Pub. Date: Sep. 18, 2008

(54) Nonwoven vascular prosthesis and process for its production

(75) Inventors: Helmut Goldmann, Tuttlingen/Donau (DE); Dennis Langanke, Tuttlingen/Donau (DE); Dietmar Probst, Tuttlingen/Donau (DE)

Correspondence Address: NATH & ASSOCIATES, 112 South West Street, Alexandria, VA, 22314, US

(73) Assignee: AESCULAP AG & CO.KG, Tuttlingen/Donau (DE)

(21) Appl. No.: 12/068,848

(22) Filed: Feb. 12, 2008

(30) Foreign Application Priority Data
Feb. 13, 2007 (DE) 10 2007 008185.7

(51) Int. Cl. A61F 2/06; B29C 41/46

(52) U.S. Cl. 623/1.28; 264/319

(57) ABSTRACT

The object of the invention is a nonwoven vascular prosthesis (1) with pleats (3) in the vessel wall (2). In a process for producing the pleated, nonwoven vascular prosthesis, the vessel wall is formed on a rod-shaped core (6) having a corrugated surface (7) corresponding to the pleats.

The invention relates to a nonwoven vascular prosthesis.

Nonwoven vascular prostheses in the form of porous tubes are already well-known. They can be made from expanded polytetrafluoroethylene (e-PTFE) and, depending on the thickness of the wall, have a stable cross-section. One suitable method for producing nonwoven vascular prostheses is by using a spraying technique, in which a solution of a polymer in a slightly liquid solvent is sprayed onto a core. The solvent evaporates as it passes along the spraying path, so that polymer fibers, which are still tacky, are deposited onto the core; these bond with each other to form a three-dimensional fibrous structure. The advantage of this spraying technique is that curved vascular prostheses can be produced if an appropriately curved core is used. These types of curved, nonwoven vascular prostheses are described in DE-A-101 62 821.8, for example.

Vascular prostheses having curved sections and straight sections, or sections having different curved segments, are frequently needed. It is difficult to make up these types of vascular prostheses from prefabricated individual sections. Therefore, it is desirable to produce a nonwoven vascular prosthesis, which can be bent in the desired manner, without any risk of collapse.

This object can be achieved by providing a nonwoven vascular prosthesis with pleats in the vessel wall.

Pleats are already used in textile vascular prostheses, especially woven or knitted vascular prostheses. They can be produced by forming circulating, crosswise folds, compacting the folds in the axial direction, and fixing the crosswise folds in place. The pleats in textile vascular prostheses consist of many tightly packed, accordion-like folds having relatively sharp edges. The pitch of the helically running pleats in textile vascular prostheses is usually less than a millimeter.

This form of pleating is not possible with nonwoven vascular prostheses simply because of the three-dimensional fibrous structure.

According to the present invention, the pleats preferably are in the form of waves. There are gentle transitions between the peaks and troughs of the waves, at least at the outer surface. Unlike the pleats in textile vascular prostheses, this invention does not provide for any compacting in the lengthwise direction. This means that the vascular prosthesis of the present invention is only slightly extensible in the lengthwise direction, and only then as a function of the elasticity of the material used for the wall. The longitudinal forces that occur during implantation and when the device is in situ in the body mean that the extensibility is usually a maximum of 10%.

The cross-section of the vascular prosthesis of the present invention is extremely stable and can be bent acutely, without any danger of the prosthesis wall collapsing, unlike similar nonwoven vascular prostheses which are not pleated.

The pleated vascular prosthesis of the present invention is preferably porous, i.e. the wall of the vessel is porous. If required, this can be sealed using a resorbable impregnating agent. Like existing nonwoven vascular prostheses, the prosthesis of the present invention is preferably made from a web, particularly a sprayed web. Polyurethane is particularly suitable for use as the material in the wall. Thermoplastic polyurethane, i.e. linear polyurethane, particularly a polyurethane that is soluble in solvents, is the preferred material. The porosity, which is defined in terms of the air permeability, is preferably 1 to 150 ml of air per square centimeter per minute at a pressure differential of 1.2 KPas.

The pleats may be in the form of circulating grooves, but pleats which run helically along the vessel wall are preferred. Furrows in the pleats, particularly wave troughs, are preferably formed as grooves. In one embodiment of the invention, the furrows are formed by constricted zones. In the region of the furrows, particularly the constricted zones, the vessel wall may have a denser construction and, in particular, may be compacted. The compaction of the vessel wall in the region of the furrows is preferably 10 to 60% and particularly 20 to 50% of the wall thickness outside the furrows. The wall material in one embodiment of the invention in the region of the furrows is compacted in the radial direction, and is preferably only 40 to 90%, particularly 50 to 80%, of the wall thickness outside the compacted area.

The diameter of the vascular prosthesis of the present invention may lie within the normal range. The internal diameter is preferably 2 to 40, particularly 4 to 12 mm. Even with smaller internal diameters of less than 10 mm, the vascular prosthesis of the present invention exhibits particularly favorable characteristics.

The vessel wall may have a thickness of 0.2 to 1 mm, particularly 0.4 to 0.6 mm. The difference between the wave peaks and troughs in the pleats, i.e. the depth of the grooves, is preferably 0.2 to 1 mm, and particularly 0.4 to 0.6 mm. The axial distance between the peaks, particularly the pitch of a helical pleat, is preferably in the region of 1 to 5 mm, preferably 1.5 to 3.5 mm, and particularly 2 to 3 mm. With prostheses having an internal diameter of less than 10 mm, the axial distance is preferably higher, particularly above 2.5 mm. With prostheses having an internal diameter of 10 mm and above, the distance is preferably lower, particularly below 2.5 mm. This type of arrangement results in excellent cross-sectional stability in a bent state.

The invention also relates to a process for producing the pleated, nonwoven vascular prosthesis of the present invention. The production process involves forming the vessel wall on a rod-shaped core having a corrugated surface corresponding to the pleats. A rod-shaped core with a helically encircling corrugated construction on its surface is preferred, so that the vascular prosthesis exhibits a correspondingly helically running pleated arrangement.

Various possibilities are available for forming the pleats. In one embodiment of the invention, a prefabricated, unpleated, nonwoven vascular prosthesis is pushed onto the rod-shaped core to produce the pleats. The pleats are then formed by heat treatment, which causes a reduction in size of the cross-section. The prefabricated vascular prosthesis may exhibit an internal diameter which corresponds to the external diameter of the rod-shaped core, or it may be slightly larger. It is also possible to push a prefabricated, tubular vascular prosthesis onto the core, which increases the diameter. Particularly advantageous is a prefabricated vascular prosthesis which is capable of shrinking, so that it can be shrunk onto the corrugated rod.

The pleats can be shaped by permanent narrowing of the vascular prosthesis in the region of the wave troughs of the pleats. This can be achieved using the shrinkage effect already mentioned. It is also possible to constrict the prefabricated vascular prosthesis in the region of the wave troughs and to fix this arrangement in place using suitable methods. Constriction can be effected by winding a yarn around the tubular vascular prosthesis so that it corresponds to the inclination of the helixes of the pleats in the region of the furrows, so that they are pressed into the furrows of the rod-shaped core and are fixed in place. Shrinking can also be combined with mechanical constriction. Once the pleats formed have been fixed, the rod can be removed from the pleated vascular prosthesis.

Separating the rod from the pleated vascular prosthesis can be facilitated by coating the surface of the rod with a slippery layer. Such a slippery layer may consist of a slippery, ductile mass, or else it may be in the form of a film-like intermediate layer.

In one embodiment of the invention, the nonwoven vascular prosthesis may be formed directly on the rod-shaped core. This can be done by producing the pleated vessel wall directly on the rod-shaped core. Once again, the spraying technique is suitable in this case, particularly the spray web-forming technique.

Particularly when producing the vessel wall directly on the rod-shaped, corrugated core, according to a preferred embodiment, cores having diameters that can be reduced in size or cores which can be taken apart, are particularly suitable. It is therefore possible to manufacture the core so that it is made up of several parts. For example, a cylindrical rod can be used to form the core, which is combined with a helix of the relevant size, which can be pushed on and off.

Other characteristics of the invention can be seen in the following diagrams, together with the dependent claims. The characteristics may stand alone or else they may be combined with each other.

The diagrams show

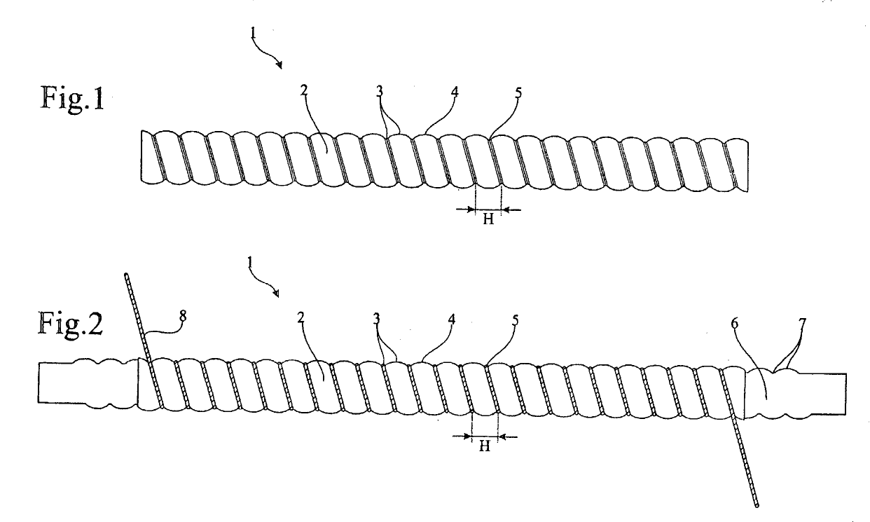

FIG. 1: one embodiment of a pleated nonwoven vascular prosthesis claimed in the present invention, and

FIG. 2: a production stage, in which the prosthesis is still located on a rod-shaped core.

The embodiment shown in FIG. 1 is a nonwoven vascular prosthesis 1 made from a sprayed polyurethane web, in which the vessel wall 2 is formed as a porous, sprayed web and which exhibits a helically shaped, corrugated pleated arrangement 3. The prosthesis wall 2 consists of a multiplicity of polyurethane fibers which create a three-dimensional, porous structure and which are bonded together. The porosity corresponds to an air permeability of 30 ml of air per square centimeter per minute at a pressure differential of 1.2 KPas. The pleats of the vascular prosthesis have a pitch H of 2.7 mm. The corrugated construction of the pleats 3 is asymmetrical. The convex arches 4 forming the peaks of the waves have a larger radius than the concave grooves 5 forming the troughs of the waves.

The thickness of the vascular prosthesis wall is 0.5 mm. The clear internal diameter of the vascular prosthesis measures 5 mm. The external diameter is 6.0 mm in the region of the wave peaks and 5.5 mm in the region of the furrows.

The vascular prosthesis can be bent acutely without collapsing. The resilience of the pleated vascular prosthesis is considerably greater on radial compression than that of an unpleated vascular prosthesis.

As FIG. 2 shows, the prosthesis of the present invention can be produced by pushing a prefabricated, porous, nonwoven vascular prosthesis in the form of a cylindrical tube onto a rod 6 having a helical corrugated construction 7 corresponding to the desired pleating arrangement. The prosthesis is heated for a short period of time and molds to roughly the corrugated shape of the rod by shrinking in the cross-section, whereby the helix shape of the rod is visible on the upper side of the tube. A yarn 8, made from polyester, for example, is then wound around the wall of the prosthesis so that it corresponds to the helix shape of the rod, and the tube wall is pressed helically into the corrugated furrows of the rod. The tube takes on the corrugated shape of the rod by carrying out heat treatment at 50° C., and this is retained on cooling.

Once the yarn 8 has been removed, the rod can be pulled easily out of the pleated prosthesis.

In another embodiment of the process to produce the prosthesis of the present invention, a core, whose diameter can be changed or which can be taken apart, can be used. With this embodiment, cores are provided, in which wedge-shaped or conical internal sections can be removed from the core, reducing the diameter at the same time. Alternatively, the helix is arranged so that it can be pushed along on a rod-shaped, cylindrical core. In particular, when using such cores, the prosthesis can be produced with pleats directly on the core, by immersion or cumulative spraying.

1. A nonwoven vascular prosthesis (1) with pleats (3) in the vessel wall (2).

2. The nonwoven vascular prosthesis (1) wherein the pleats (3) are in the form of waves.

3. The nonwoven vascular prosthesis as claimed in claim 1, wherein the vessel wall (2) is porous and may be sealed using a resorbable impregnating agent if required.

4. The nonwoven vascular prosthesis as claimed in claim 1, wherein the vessel wall (2) is formed from a web, particularly a sprayed web.

5. The nonwoven vascular prosthesis as claimed in claim 1, wherein the vessel wall (2) is made from polyurethane.

6. The nonwoven vascular prosthesis as claimed in claim 5, wherein the polyurethane is a thermoplastic polyurethane, particularly a polyurethane that is soluble in a solvent.

7. The nonwoven vascular prosthesis as claimed in claim 1, wherein the pleats (3) have grooves (5), which preferably run helically along the vessel wall (2).

8. The nonwoven vascular prosthesis as claimed in claim 1, wherein furrows, particularly grooves (5), are formed by constricted zones in the pleats (3).

9. The nonwoven vascular prosthesis as claimed in claim 8, wherein the vessel wall (2) in the region of the furrows (5) is compacted, particularly by constricted zones.

10. The nonwoven vascular prosthesis as claimed in claim 1, wherein the internal diameter of the prosthesis measures 2 to 40 mm, and particularly 4 to 12 mm.

11. The nonwoven vascular prosthesis as claimed in claim 1, wherein the thickness of the vessel wall (2) measures 0.2 to 1 mm, and particularly 0.4 to 0.6 mm.

12. The nonwoven vascular prosthesis as claimed in claim 1, wherein the pleats (3) have a groove depth of 0.2 to 1 mm, and particularly 0.4 to 0.6 mm.

13. A process for producing the pleated, nonwoven vascular prosthesis as claimed in claim 1, wherein the vessel wall is formed on a rod-shaped core having a corrugated surface corresponding to the pleats.

14. The process as claimed in claim 15, wherein a rod-shaped core having a helically encircling, corrugated construction on its surface is used.

15. The process as claimed in claim 13, wherein a prefabricated, unpleated, nonwoven vascular prosthesis is pushed onto the rod-shaped core to produce the pleats and the pleats are formed in particular by heat treatment, which causes a reduction in size of the cross-section.

16. The process as claimed in claim 13, wherein the prefabricated nonwoven vascular prosthesis on the rod-shaped core is constricted in the wave troughs of the corrugated surface, and the constricted zones are fixed in place.

17. The process as claimed in claim 13, wherein a prefabricated vascular prosthesis, that has been reversibly pre-stretched in the cross-section, is pushed onto the rod-shaped core and shrunk onto it.

18. The process as claimed in claim 13, wherein a slippery intermediate layer, in particular a film, is applied to the rod-shaped core, in particular between the rod-shaped core and the prefabricated vascular prosthesis.

19. The process as claimed in claim 13, wherein the prosthesis is produced with pleats by spraying a solution of polyurethane onto the rod-shaped core to form a sprayed web with a corrugated surface.

20. The process as claimed in claim 13, wherein a core, having a corrugated surface and a cross-section that can be reduced in size, is used; the cross-section of the core can be reduced to facilitate removal of the pleated vascular prosthesis.

21. The process as claimed in claim 13, wherein the corrugated surface of the core is formed by a helix, which is wound onto a rod, and which is preferably removable in the lengthwise direction.
